(19) United States
(12) Patent Application Publication
(10) Pub. No.: US 2015/0250523 A1
(43) Pub. Date: Sep. 10, 2015

(54) METHOD FOR TREATING VARICOSE VEINS AND INTRALUMINAL DEVICE USED IN SUCH METHOD

(71) Applicant: TERUMO KABUSHIKI KAISHA, Tokyo (JP)

(72) Inventors: Katsuhiko SHIMIZU, Ashigarakami-gun (JP); Katsunori EBATA, Ashigarakami-gun (JP)

(73) Assignee: TERUMO KABUSHIKI KAISHA, Tokyo (JP)

(21) Appl. No.: 14/203,501

(22) Filed: Mar. 10, 2014

(51) Int. Cl. A61B 18/00; A61B 18/14; A61B 18/24; A61B 18/08

(52) U.S. Cl. CPC A61B 18/00; A61B 18/082; A61B 18/1492; A61B 18/24; A61B 2018/00404; A61B 2018/0063; A61B 2018/00285

(57) ABSTRACT

A method of treating a varicose vein involves contacting the inner wall of the vein with a contact member, withdrawing an outer sheath relative to a self-expanding member positioned inside the outer sheath while the contact member remains in contact with the inner wall of the vein to cause a first spasm in the vein that decreases the vein inner diameter, relatively moving the outer sheath and the self-expanding member so that the self-expanding member is located distally beyond the distal end of the outer sheath so that the self-expanding member self-expands outwardly into contact with the inner wall of the vein, and moving the catheter and the self-expanding member relative to the vein while the self-expanding member is in contact with the inner wall of the vein to cause a second spasm in the vein, and applying energy to the inner wall of the collapsed vein.

### TECHNICAL FIELD

The invention pertains to a method of treating veins and an intraluminal device used in performing such method. More specifically, the invention involves a method of treating varicose veins and an intraluminal device for such method

### BACKGROUND DISCUSSION

There are a variety of conditions in which it is desirable to stop the circulation through particular blood vessels. For example, one treatment for varicose veins involves reducing or stopping blood circulation through the affected vein. Varicose veins have been treated by injecting a sclerosant which is an injectable irritant that causes inflammation and subsequent fibrosis to close off the lumen of the vein. It is rather easy for a sclerosant to become diluted because of the existence of the blood. Another technique which has been used involves stimulating the inner wall of the vein to decrease the inner diameter of the vein and then inject a sclerosant to close the blood vessel. Decreasing the inner diameter maintains the concentration of sclerosant because of the small amount of blood.

Other proposals for treating varicose veins have involved using ablation devices. Here, tumescent local anesthesia is used to create a spasm in the vein followed by the use of ablation devices. The tumescent local anesthesia presents challenges in that it is oftentimes difficult to know the exact source of the problems for treating varicose veins and so it is sometimes desirable to treat the entire vein. But it is difficult to create a spasm along the length of the vein in a uniform matter. Also, spasm is typically only affective for part of the length of the vein using the known techniques and so multiple spasm must be created.

Various publications describe other known method and apparatus. For example, U.S. Pat. No. 7,862,575 describes a known vascular ablation device, U.S. Pat. No. 6,402,745 describes an intravenous surgical instrument used to collapse circulatory vessels, U.S. Pat. No. 7,396,355 describes an apparatus for applying energy to shrink a vein, and U.S. Pat. No. 7,833,240 describes an atherectomy catheter that can be used to remove material that is stenosing the lumen in a tubular organ.

### SUMMARY

According to one aspect of the disclosure here, a method of treating a varicose vein comprises: inserting an assembly into the vein, the assembly being comprised of: an outer sheath that includes a contact member and possesses a distal end; a catheter movably positioned inside the outer sheath and possessing a distal end at which is located a self-expanding member, the self-expanding member possessing a distal end; and an elongated body positioned inside the catheter and possessing a distal end at which is located an energy emitting member, the energy emitting member possessing a distal end; moving the assembly to a treatment site in the vein; moving the contact member into contact with an inner wall of the vein; withdrawing the outer sheath relative to the catheter and the self-expanding member while the contact member remains in contact with the inner wall of the vein to cause a first spasm in the vein that decreases an inner diameter of the vein; the withdrawing of the outer sheath relative to the catheter and the self-expanding member causing the distal end of the self-expanding member to be located distally beyond the distal end of the outer sheath so that the self-expanding member self-expands outwardly into contact with the inner wall of the vein; moving the catheter and the self-expanding member in a proximal direction relative to the vein while the self-expanding member is in contact with the inner wall of the vein to cause a second spasm in the vein that further decreases the inner diameter of the vein so that the vein collapses; applying energy from the energy emitting member to the inner wall of the collapsed vein to cause the vein to uniformly and immediately occlude; and withdrawing the assembly from the vein.

According to another aspect, a method of treating a varicose vein involves: inserting an assembly into the vein, the assembly being comprised of: an outer sheath that includes a contact member and possesses a distal end; a self-expanding member positioned inside the outer sheath, the self-expanding member possessing a distal end and being outwardly expandable when the distal end of the self-expanding member is exposed distally beyond the distal end of the outer sheath; and an energy emitting member; moving the assembly in the vein to position the assembly at a desired place in the vein; contacting an inner wall of the vein with the contact member; moving the outer sheath relative to the inner wall of the vein while the contact member is in contact with the inner wall of the vein to produce a first spasm in the vein that decreases an inner diameter of the vein; relatively moving the outer sheath and the self-expanding member while the contact member is in contact with the inner wall of the vein to expose the distal end of the self-expanding member distally beyond the distal end of the outer sheath such that the self-expanding member self-expands outwardly into contact with an inner wall of the vein at a position distal of a position at which the contact member is in contact with the inner wall of the vein; decreasing an inner diameter of the vein and causing the vein to collapse by moving the expanded self-expanding member relative to the vein while the expanded self-expanding member is in contact with the inner wall of the vein; and occluding the vein by applying energy from the energy emitting member to the inner wall of the vein which has collapsed.

### BRIEF DESCRIPTION OF THE DRAWINGS

Additional details, characteristics and aspects of the method and device disclosed here will become more apparent from the following detailed description considered with reference to the accompanying drawing figures.

FIG. 1 is a schematic view of one version of an assembly or intraluminal device representing an example of the assembly or intraluminal device disclosed here.

FIGS. 2A-2D illustrate sequential operational aspects of the intraluminal device and the varicose vein treatment method, representing an example of the treatment method disclosed here.

FIGS. 3A-3E illustrate embodiments of the self-expanding member forming a part of the intraluminal device shown in FIG. 1.

FIGS. 4A-4E illustrate sequential operational aspects of a varicose vein treatment method disclosed here as another example of the treatment method disclosed here.

FIGS. 5A-5F illustrate different embodiments of the energy-emitting member forming a part of the assembly or intraluminal device illustrated in FIG. 1.

FIG. 6 illustrates an intraluminal device according to a another embodiment.

FIG. 7A is a cross-sectional view of a distal end portion of the intraluminal device shown in FIG. 6 taken along the section line 7A-7A, FIG. 7B is a similar cross-sectional view after the self-expanding member has expanded outwardly into contact with the inside wall of the vein, and FIGS. 7C and 7D are cross-sectional views of a part of the distal end portion of the intraluminal device shown in FIG. 6 illustrating different embodiments that include a plurality of cutting members.

FIG. 8A is a view similar to FIG. 7A illustrating a variation on the device and method shown in FIGS. 6 and 7A-7B, and FIGS. 8B and 8C illustrate subsequent operational aspects of the method and the device of FIG. 8A.

FIG. 9A is an enlarged illustration of the portion identified 9A in FIG. 8B, and FIG. 9B is an enlarged illustration of the portion designated 9B and FIG. 8C.

FIGS. 10A and 10B illustrate the outwardly expandable member forming a part of the intraluminal device illustrated in FIG. 6, where in FIG. 10A illustrates the self-expanding member before expansion and FIG. 10B illustrates the self-expanding member after expansion.

FIG. 11 illustrates another embodiment of the intraluminal device that is similar to the device shown in FIG. 6, but with the addition of an energy emitting member.

FIG. 12 is a cross-sectional view illustrating the intraluminal device positioned in a vein.

FIG. 13 is a cross-sectional view showing the intraluminal device during one operational aspect.

FIG. 14A is an enlarged view of the portion identified as 14A in FIG. 13.

FIG. 14B is similar to FIG. 14A, but depicting the energy emitting member at a different position.

FIG. 15 illustrates the energy emitting member shown in FIG. 5F positioned in a vein during an operational aspect of the intraluminal device.

FIG. 16A illustrates the energy emitting member shown in FIG. 5D connected to the end of the rotatable elongated member.

FIG. 16B illustrates the energy emitting member shown in FIG. 16A positioned in a vein during an operational aspect of the intraluminal device.

### DETAILED DESCRIPTION

Generally speaking, the device and method disclosed here are used to treat veins, including varicose veins. Varicose veins in the lower limb are most prevalent, though they also occur in pelvic and ovarian and spermatic cord veins. The treatment here seeks to close or occlude the affected vein. In one respect, this is accomplished by bringing a member into contact with the inner wall of the vein and moving the member along the vein to cause a spasm that decreases the inner diameter of the vein (vein lumen), and applying energy to the collapsing vein to fuse together the vein inner wall so that the lumen in the vein is occluded. It is also possible to treat the vein by contacting the inner wall of the vein in a manner causing damage to the inner wall of the vein so that blood clots form which occlude the vein (vein lumen). The method disclosed here thus involves the use of a decreased level or amount of a tumescent local anesthesia, a sclerosant, or a combination of a tumescent local anesthesia and a sclerosant.

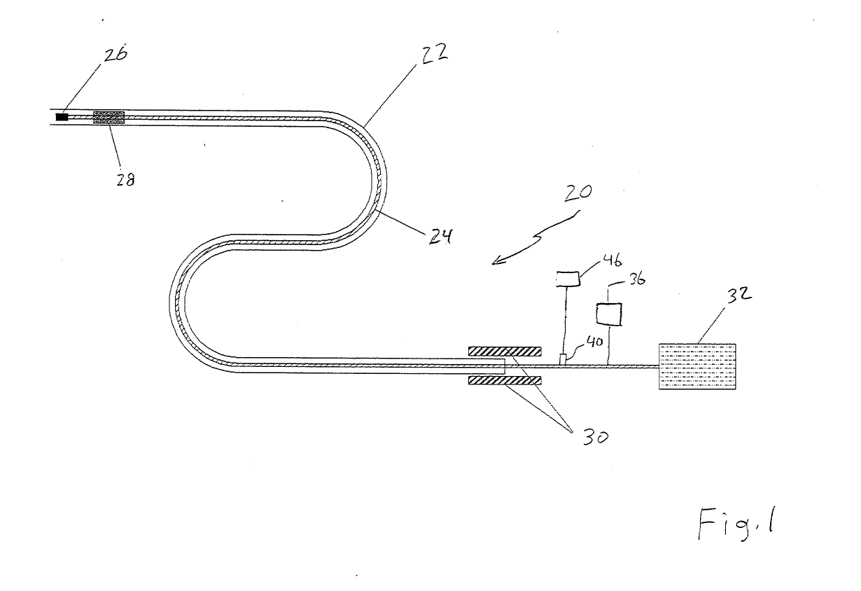

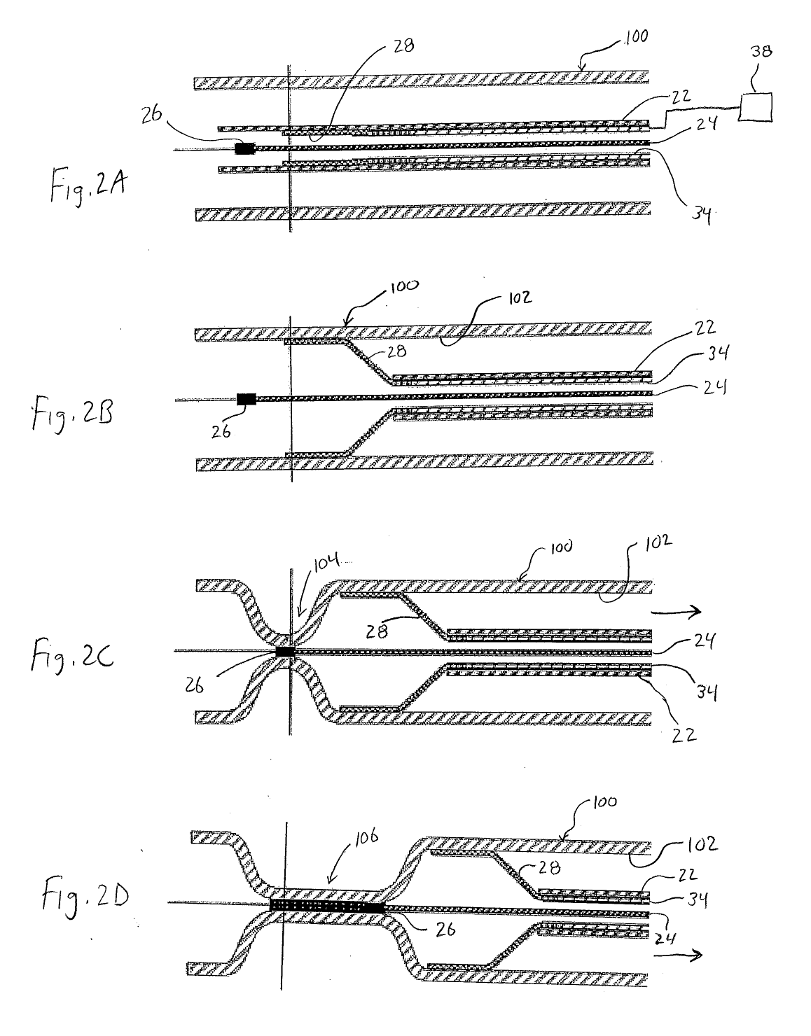

FIG. 1 illustrates, somewhat schematically, an assembly or intraluminal device disclosed here by way of example for caring out treatment methods as described below. The assembly or intraluminal device 20 includes an outer sheath 22, and elongated member 24 positioned inside the outer sheath 22, an energy emitting member 26 fixed at the distal end of the elongated member 24 and surrounded by the outer sheath 22, and a self-expanding member 28 also positioned inside and surrounded by the outer sheath 22. As will be described in more detail below, the self-expanding member 28 is fixed at the distal end of an inner sheath (catheter) so that the self-expanding member 28 and the inner sheath are movable together as single unit. The energy emitting member 26 fixed at the distal end of the elongated member 24 also moves together as a unit with the elongated member 24. To avoid excessive details, the inner sheath to which the self-expanding member 28 is connected is not illustrated in FIG. 1, but such inner sheath will be described in more detail with reference to FIGS. 2A-2B.

As further illustrated in FIG. 1, the intraluminal device 20 includes a moving device 30 operatively connected to the outer sheath 22 to axially move the outer sheath 22 relative to the elongated member 24 and the associated energy emitting member 26. That is, operation of the moving device 30 produces relative axial movement between the outer sheath 22 and the elongated member 24/energy emitting member 26. The moving device 30 also axially moves the outer sheath 22 relative to the inner sheath to which the self-expanding member 28 is fixed. The operation of the moving device 30 thus also produces relative axial movement between the outer sheath 22 and the inner sheath/self-expanding member 28. As will be described in more detail below, axially moving the outer sheath 22 through operation of the moving device 30 exposes the emitting member 26 and/or the self-expanding member 28 beyond the distal end of the outer sheath 22. The moving device 30 can be any known type of moving device for actually moving the outer sheath 22. For example, a rack and pinion arrangement can be employed. It is also possible to move the outer sheath 22 by hand.

FIG. 1 further illustrates that the intraluminal device 20 includes an energy source 32 connected to the energy emitting member 26. The energy emitting member 26 can take one of several forms, and the energy source 32 is appropriately selected based on the energy emitting member 26. For example, the energy emitting member 26 can be a laser, in which case the energy source 32 is a laser source. Alternatively, the energy emitting member 26 can be a single/mono-polar or bi-polar electrode in which case the energy source 32 is a RF-generator. Further yet, the energy emitting member 26 can be in the form of a heater, a coil, steam or high temperature fluid, in which case the energy source 32 is a heater, a thermoelectric-generator, a steam-generator or a hot-water bath, respectively.

FIG. 2A is an enlarged illustration of the distal end portion of the intraluminal device 20 shown in FIG. 1. The distal end portion of the intraluminal device is positioned in a vein 100. FIG. 2A illustrates the elongated member 24 and the associated energy emitting member 26 positioned inside the outer sheath 22. Also shown is the inner sheath 34 (catheter) to which the self-expanding member 28 is fixed so that the self-expanding member 28 and the inner sheath 34 move together as a unit. The self-expanding member 28 and the inner sheath 34 are positioned inside the outer sheath 22 (i.e., are surrounded by the outer sheath 22) when the distal end of portion of the intraluminal device is introduced into the vein. The outer sheath 22, the inner sheath 34 and the elongated member 24 are preferably coaxially arranged or arranged offset.

In this illustrated embodiment of the intraluminal device, the self-expanding member 28 is an annular member that is generally cylindrical in shape. The self-expanding member 28 is configured and/or made of a material which maintains the self-expanding member 28 in the non-expanded state shown in FIG. 2A while the self-expanding member 28 is surrounded by and held within the outer sheath 22. But when the position of the outer sheath 22 relative to the self-expanding member 28 changes so that the self-expanding member 28 is no longer covered by and held inside the outer sheath 22, the self-expanding member 28 automatically expands outwardly (i.e., self-expands outwardly).

Set forth next is an explanation is a manner of using or operating the intraluminal device 20 (assembly) for carrying out one example of a method disclosed here. To begin, the distal end portion of the intraluminal device 20 is inserted into a vein of interest (e.g., an affected varicose vein). The intraluminal device or assembly is then moved along the vein until reaching the target sight or treatment location. FIG. 2A illustrates the distal end portion of the intraluminal device located at the treatment site in the vein 100. After the intraluminal device is properly positioned, the outer sheath 22 is axially withdrawn or axially moved in the proximal direction (to the right in FIG. 2A). More specifically, the outer sheath 22 is axially moved in the proximal direction relative to the inner sheath 34 and the attached self-expanding member 28, and relative to the elongated member 24 and the attached energy emitting member 26. Relative movement thus occurs between the outer sheath 22 and the inner sheath 34/self-expanding member 28, and between the outer sheath 22 and the elongated member 24/energy emitting member 26. This relative movement results in the self-expanding member 28 being positioned distally beyond the distal end of the outer sheath 22 as illustrated in FIG. 2B. In this way, the self-expanding member 28 (distal end portion of the self-expanding member) self-expands or automatically expands radially outwardly into contact (direct contact) with the inner wall 102 (inner surface) of the vein 100. As a result of the proximal relative movement of the outer sheath 22, the energy emitting member 26 is also exposed distally beyond the distal end of the self-expanding member 28 as shown in FIG. 2B. That is, the energy emitting member 26 is positioned distally beyond the distal end of the self-expanding member 28 so that the energy emitting member 26 is no longer covered.

Next, the outer sheath 22, the inner sheath 34 and the elongated member 24 are moved in the proximal direction indicated by the arrow in FIG. 2C. As a result, the self-expanding member 28 which is in contact with the inner wall or inner surface 102 of the vein 100 moves in the proximal direction. The energy emitting member 26 which is located distal to, or in front of, the distal-most end of the self-expanding member 28 also moves in the proximal direction indicated by the arrow. During the proximal movement of the self-expanding member 28 and the energy emitting member 26, the relative positions of the energy emitting member 26 and the self-expanding member 28 is maintained. That is, while the self-expanding member 28 and the energy emitting member 26 are being withdrawn toward the proximal direction, the energy emitting member 26 remains distally in front of the distal-most end of the self-expanding member 28.

As the self-expanding member 28 is moved in the rearward direction while in contact with the inner wall 102 of the vein 100, the vein 100 experiences spasm causing the inner diameter of the vein to decrease uniformly. That is, by the virtue of the annular or cylindrical outer surface of the self-expanding member 28 contacting the inner wall 102 of the vein 100 while moving rearwardly or in the proximal direction, the entire circumferential extent of the inner wall 102 of the vein 100 is in contact with the self-expanding member 28 and so the diameter of the vein decreases uniformly. When the self-expanding member 28 contacts the inner wall 102 of the vein while moving rearwardly or in the proximal direction, the self-expanding member 28 can be rotated around the longitudinal axis of the self-expandable member 28 to increase contacts between the inner wall 102 and the self-expandable member 28. Spasm is to the inwardly moving or collapsing inner wall 102 of the vein 100. This movement causes more spasm uniformly.

FIG. 2C illustrates a region 104 of the vein 100 where the inner diameter of the vein has decreased uniformly (the vein has collapsed uniformly) due to the spasm created by the self-expanding member 28 being moved along the inner wall 102 of the vein. The energy emitting member 26 is placed in the uniformly collapsed vein distally beyond the distal end of the self-expanding member 28. When the energy emitting member 26 is in the region 104 of the vein where the inner diameter of the vein has decreased uniformly, energy is sent to the energy emitting member 26, and such energy is applied to the vein and the protein in the vessel is heat-denatured and the vein thus becomes occluded.

The outer sheath 22, the inner sheath 34 and the elongated member 24 continue to be moved in the rearward or proximal direction as indicated by the arrow in FIG. 2D. This causes the self-expanding member 28 which is in contact with the inner wall or inner surface 102 of the vein 100 to continue moving in the rearward or proximal direction together with the energy emitting member 26. As a result, the uniform decrease in the inner diameter of the vein 100 proceeds in the axial rearward direction, and the energy emitting member 26 continues to emit energy, either in a continuous manner or in a pulsative manner (i.e., a manner in which energy is alternatively supplied and not supplied), thus causing the continued uniform and immediate decrease in the size of the vein. In addition, the protein in the vessel is heat-denatured. The vein thus becomes occluded as indicated at 106.

The above operation continues until the entirety of the vein, or the desired axial extent of the vein, is uniformly and immediately occluded as described above. After the desired axial extent of the vein is occluded, the assembly or intraluminal device is removed from the vein.

In the embodiment described above, the axial position of the distal end of the self-expanding member 28 relative to the energy emitting member 26 may be adjusted in that the elongated member 24 to which the energy emitting member 26 is fixed, and the inner sheath 34 to which the self-expanding member 28 is fixed, can be made relatively movable. This can be accomplished by, for example, connecting the elongated member 24 to a respective axial moving device 36 as schematically illustrated in FIG. 1, and connecting the inner sheath 34 to a respective moving device 38 as schematically illustrated in FIG. 2A. The moving or driving devices 36, 38 can be any known arrangement for axially moving the elongated member 24 and the associated energy emitting member 26 independently of axial movement of the inner sheath 34 and the associated self-expanding member 28. A rack and pinion arrangement is an example of the moving or driving devices 36, 38. The outer sheath 22, the inner sheath 34 and the elongated member 24 can be connected in accurate positional relation to one another, for example using a suitable connector. The moving device 36, 38 can be a manually operated moving device or an automatically operated moving device.

Alternatively, it is also possible to fix the position of the self-expanding member 28 relative to the energy emitting member 26 so that during the operation illustrated in FIGS. 2A-2D, the relative position of the self-expanding member 28 and the energy emitting member 26 does not change. This can be accomplished by, for example, fixing the elongated member 24 and the inner sheath 34 to one another so that they both move together as a unit. The elongated member 24 can be fixed axially along the inner lumen of the inner sheath and is allowed to move back and forth axially.

The distance between the distal-most end of the self-expanding member 28 and the distal-most end of the energy emitting member is preferably 1 mm-100 mm. Thus, if the position of the self-expanding member 28 relative to the energy emitting member 26 is fixed, the positional relation of the self-expanding member 28 relative to the energy emitting member 26 satisfies the distance range mentioned above. If the self-expanding member 28 and the energy emitting member 26 are relatively movable as mentioned above, the relative movement is preferably performed or controlled to maintain the distance range discussed above. The energy emitting member 26 is configured to automatically emit energy when the energy emitting member 26 is disposed in the collapsed region of the vein.

If the contact of the self-expanding member against the inside wall or inside surface 102 of the vessel 100 during rearward movement of the self-expanding member 28 does not create sufficient spasm to cause the energy emitting member to occlude the vessel, it is possible to provide the elongated member 24 with an aspiration port 40 as schematically illustrated in FIG. 1. The aspiration port 40 can be connected to a vacuum source 46 so that operation of the vacuum source 46 will draw a vacuum in the vein that will help collapse the inner wall 102 of the vessel 100 so that the energy emitting member 26 can apply energy to the inner wall 102 of the vein 100 to uniformly and immediately occlude the vein. The distal end portion of the elongated member 24 is provided with one or more openings such that suction applied at the aspiration port 40 draws-in the inner wall 102 of the vein. The outer sheath 22 has a distal opening, and can also be provided with one or more openings on the lateral surface of the distal end. Aspiration can then be made through these openings.

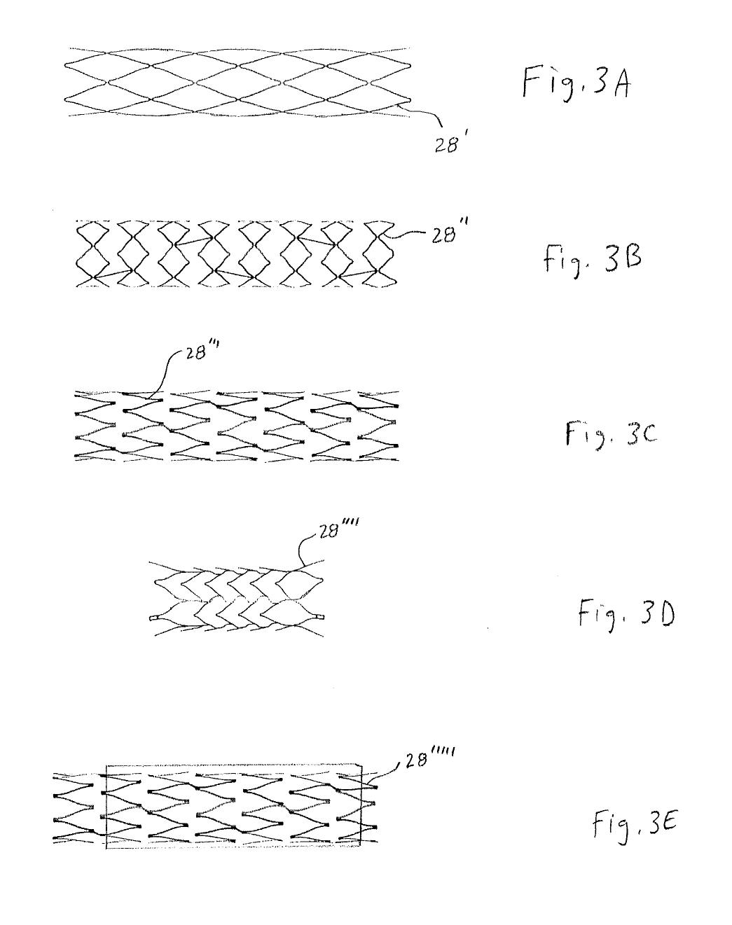

The self-expanding member 28 can take a variety of forms or shapes. Several examples are illustrated in FIGS. 3A-3E.

The self-expanding member can be a closed-cell self-expanding member 28′ such as illustrated in FIG. 3A. Alternatively, the self-expanding member can be an open-cell self-expanding member 28″ such as illustrated in FIG. 3B. FIG. 3C illustrates another open-cell self-expanding member 28′″. The open-cell and closed-cell configurations shown in FIGS. 3A and 3B differ from one another in that all of the cells are closed in the closed-cell configuration while many of the individual cells in the open-cell arrangements are open.

FIG. 3D illustrates a further embodiment of a closed-cell self-expandable member 28″″. In this embodiment, both edges of the self-expandable member 28″″ is are open-cell configurations, and the middle section of the self-expandable member 28″″ is a closed-cell arrangement. This combination design can adjust the radial and scratching force to the inner vessel.

FIG. 3E illustrates a still further embodiment of the self-expanding member 28′″″. In this embodiment, the self-expanding member is an open-cell self-expanding member, and is surrounded by a cover as shown in FIG. 3E. This embodiment of the self-expanding member 28′″″ provided with the cover is advantageous in that inflection points on the self-expanding member (i.e., the inflection points on the part of the self-expanding member 28′″″ underlying the cover are covered and thus do not tend to get hung-up through contact with the inside wall of the vein 100 while the self-expanding member 28′″″ is being moved rearwardly while in contact with the inside wall 102 of the vein 100. Another benefit associated with this covered version of the self-expanding member 28′″″ shown in FIG. 3E is that the cover tends to stop blood flow, assuming the cover is made out of an appropriate material to stop blood flow during use.

Examples of the material for forming the self-expandable member 28, the outer sheath 22 and the inner sheath 34 include metals and resins. Examples of the metals include pseudo-elastic alloys (inclusive of superelastic alloys) such as Ni—Ti alloys, shape memory alloys, stainless steels (e.g., all types of SUS, such as SUS304, SUS303, SUS316, SUS316L, SUS316J1, SUS316J1L, SUS405, SUS430, SUS434, SUS444, SUS429, SUS430F, SUS302, etc.), cobalt alloys, noble metals such as gold, platinum, etc., tungsten alloys, and carbon-containing materials (inclusive of piano wire). Examples of the resins include polymer materials such as polyolefins (e.g., polyethylene, polypropylene, polybutene, ethylene-propylene copolymers, ethylene-vinyl acetate copolymers, ionomers, or mixtures thereof), polyvinyl chloride, polyamides, polyamide elastomers, polyesters, polyester elastomers, polyurethane, polyurethane elastomers, polyimides, fluoro-resins, and mixtures of them, which may be used either singly or in combination of two or more of them. The self-expandable member 28 may be composed of a multi-layer tube or the like of a composite material formed from these metals and/or resins.

As an example, the elongated member 24 can be made as an electrical wire/cable covered with polymer jacket, a glass core or a glass clad covered with a polymer jacket.

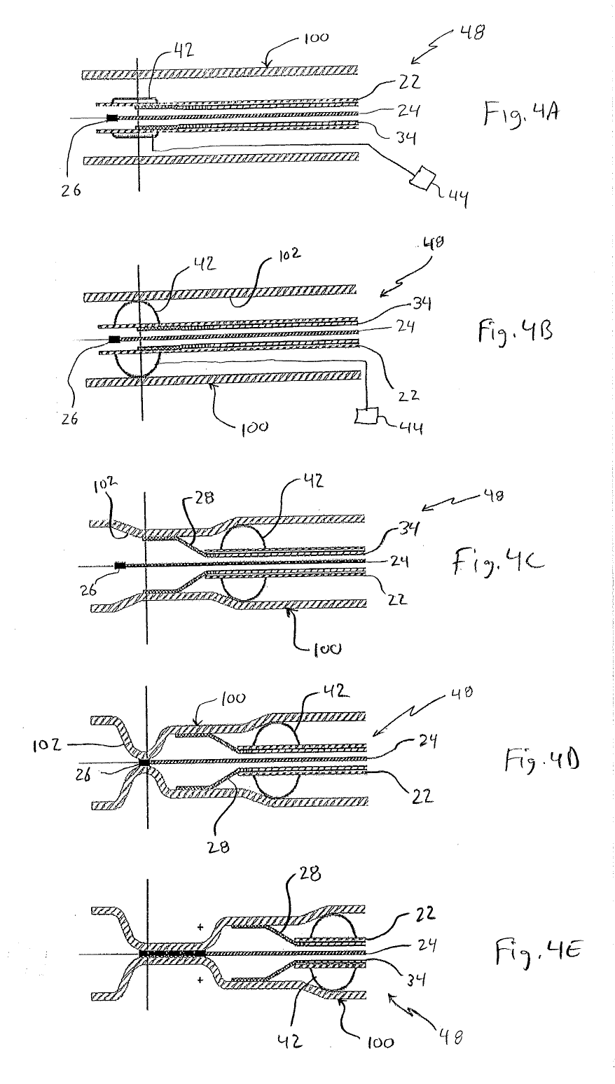

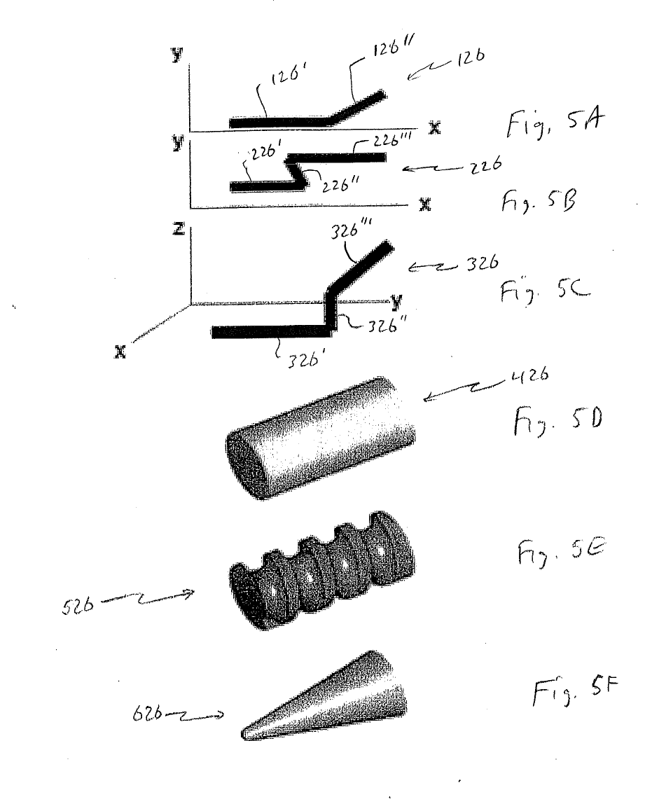

FIGS. 4A-4E illustrates another embodiment of the assembly or intraluminal device 48 representing another example of the disclosure here. This embodiment and the manner of use/operation is the same as described above with respect to the assembly or intraluminal device illustrated in FIGS. 2A-2D, except that the embodiment illustrated in FIGS. 4A-4E includes a contacting member 42 which will be described below in more detail. Other aspects of the intraluminal device and method are the same as described above in connection with FIGS. 1 and 2A-2D. Features common to both embodiments are identified by common reference numerals, and a detailed discussion of aspects of the device and manner of use/operation described above are not repeated in detail here.

As shown in FIG. 4A, the contacting member 42 is fixed to the outer sheath 22. In the illustrated embodiment disclosed by way of example, the contacting member 42 is an expandable contact member that is attached in a fluid-tight manner to the outer surface of the outer sheath 22. The interior of the contacting member 42 is connected to and communicates with a fluid source 44 which can be in the form of a pressurized fluid.

Fluid from the fluid source 44 is introduced into the contacting member 42 to outwardly expand or inflate the contacting member 42 as illustrated in FIG. 4B. This brings the contacting member 42 into contact (direct contact) with the inner surface or inner wall 102 of the vein 100 as shown in FIG. 4B.

During use or operation of the intraluminal device 48 shown in FIGS. 4A-4E, the assembly or device 48 is inserted into the intended vein (i.e., the varicose veins requiring treatment), and the distal end of the device is moved to the target site or treatment site in the vein such as illustrated in FIG. 4A. Then, as depicted in FIG. 4B, fluid is introduced into the contacting member 42 to inflate or outwardly expand the contacting member 42 into contact (direct contact) with the inner wall 102 of the vessel 100. The outer sheath 22 is then moved in the proximal or radial direction relative to the self-expanding member 28 and the energy emitting member 26 in the same manner discussed above. As the contacting member 42 moves rearwardly while in contact with the inside wall 102 of the vein 100, the vein 100 is subjected to a first spasm by virtue of the contact between the expanded contact member that is moving rearwardly or radially while in contact with the inside wall 102 of the vein. This first spasm refers to the spasm induced or created by the contacting member 42. This spasm causes the inner diameter of the vein to decrease (i.e., the vein begins to collapse). The remainder of the operation is similar to that described above. That is, the outer sheath 22 is withdrawn or moved rearwardly (in the proximal direction) so that the self-expanding member 28 is positioned distally beyond the distal end of the outer sheath 22. The self-expanding member 28 thus automatically self-expands into contact (direct contact) with the inside wall 102 of the vein 100 as shown in FIG. 4C. Rearwardly moving the self-expanding member 28, together with the energy emitting member 26 in the manner similar to that described above causes the vein 100 to experience a second spasm by virtue of the contact between the self-expanding member 28 and the inside wall 102 of the vein 100. The self-expandable member 28 can be rotated relative to the inner wall of the vein 100. This second spasm refers to the spasm induced or created by the self-expanding member 28. This second spasm causes the inside diameter of the vein 100 to further decrease or collapse as illustrated in FIG. 4D. Energy delivered to the energy emitting member 26 is then applied to the inside wall 102 of the vein 100 so that the vein is occluded uniformly and immediately. Continued rearward movement of the self-expanding member 28, the contacting member 42 and the energy emitting member 26 continues the uniform and immediate occlusion so that the occlusion continues in the axial direction as illustrated in FIG. 4E. This forms a continued length of the occluded region 106 of the vein.

In the embodiment discussed above and illustrated in FIGS. 4A-4E, the contacting member 42 is separate from and attached to the outer sheath 22. As an alternative, the contacting member 42 can be an integral part of the outer sheath, meaning that it can be a part of the outer sheath itself so that the contacting member 42 and the outer sheath 22 are integrally formed in piece at the same time.

The material forming the contacting member 42 can be the same as or different from the material forming the self-expanding member 28. According to one embodiment, the material forming the contacting member 42 is different from the material forming the self-expanding member 28. One advantage of this is that it is possible to select different materials which provides stronger or weaker contact with the inside wall 102 of the vein 100 to create more or less spasm, thus facilitating or delaying collapse (reduced inner diameter) of the vein 100. In a step of causing or creating a first spasm, the operator can examine the strength of the first spasm by the movement of the slide resistance of the device, and evaluate the responsiveness of the vein. The operator can get a sense of what the strength of the first spasm is like in the first spasm. Then the operator can decide the desired strength of the second spasm to be caused or created. In this connection, depending on the strength of the first spasm, the operator can adjust the strength of the second spasm to give the synergistic and efficient outcome of the treatment for the vein.

Examples of materials for fabricating the contacting member include thermoplastic resins such as polyolefins, polyvinyl chloride, polyamides, polyamide elastomers, polyester elastomers, polyurethane, polyesters, polyarylene sulfides, etc., silicone rubbers, and latex rubber. Particularly, stretchable (orientable) materials are preferred, and the contacting member 42 is preferably formed of a biaxially oriented material having high strength and tensile strength.

The self-expanding member 28 can be made of a material which can be employed in the case of the stent being a self-expandable stent, superelastic metals are used suitably. As the superelastic metals, superelastic alloys are used preferably. The term “superelastic alloys” used here means alloys which are generally called shape memory alloys and which show superelasticity at least at a living body temperature (around 37° C.). More preferably, superelastic alloys such as TiNi alloys containing 49 to 53 atomic % of Ni, CuZn alloys containing 38.5 to 41.5 wt % of Zn, CuZnX alloys (X=Be, Si, Sn, AI, or Ga) containing 1 to 10 wt % of X, and NiAl alloys containing 36 to 38 atomic % of Al are used. Especially preferable are the above-mentioned TiNi alloys. Mechanical characteristics of the above-mentioned alloys can be appropriately changed by adopting TiNiX alloys (X=Co, Fe, Mn, Cr, V, Al, Nb, W, B or the like) obtained by replacing a part of the TiNi alloys with 0.01 to 10.0 wt % of X, or adopting TiNiX alloys (X=Cu, Pb, Zr) obtained by replacing a part of the TiNi alloys with 0.01 to 30.0 atomic % of X, or by selection of cold work ratios or/and final heat treatment conditions. Further, the above-mentioned TiNiX alloys can be used after appropriately changing their mechanical characteristics through selection of cold work ratios or/and final heat treatment conditions. The buckling strength (yield stress when load is applied) of the superelastic alloy to be used is 5 to 200 kg/mm2 (22° C.), preferably 8 to 150 kg/mm2, and the restoring stress (yield stress when load is eliminated) of the superelastic alloy is 3 to 180 kg/mm2 (22° C.), preferably 5 to 130 kg/mm2. The term “superelasticity” used here means a property of a material such that even after deformation (bending, stretching, or compression) of the material into a region in which ordinary metal is plastically deformed at a service temperature, release of the deformation results in the material being restored substantially to its pre-deformation shape without need for heating.

In the embodiment illustrated in FIGS. 4A-4E, similar to the first embodiment described above and illustrated in FIGS. 1 and 2A-2D, the relative position of the distal end of the self-expanding member 28 and the energy emitting member 26 can be fixed or adjustable. Also, the embodiment shown in FIGS. 4A-4E can include an aspiration port on the elongated member at a position proximal to the energy emitting member 26 in a manner similar to the aspiration port 40 (and the associated fluid source 46) discussed above.

Also consistent with the discussion above, the length of the self-expanding member 28 can be in the range of 10 mm-200 mm, and the distance between the distal end of the sub-expanding member and the energy emitting member can be from 1 mm-50 mm.

In the embodiments of the intraluminal device described above, the energy emitting member 26 possesses a shape such that the energy emitting member 26 contacts the inner wall 102 of the vein 100 along a line. In addition, to avoid adherence of the energy emitting member 26 to the inner wall 102 of the vein 100 during operation, it is possible to rotate the energy emitting member 26. This can be accomplished by rotating the elongated member 24 to which the energy emitting member 26 is fixed. This rotation can be accomplished by suitably configuring the moving device 36 schematically illustrated in FIG. 1 so that the moving device 36 is able to rotate the elongated member 24 and the energy emitting member 26.

FIGS. 5A and 5F illustrate different configurations for the shape of the energy emitting member. FIG. 5A illustrates that the energy emitting member 126 includes a first straight portion 126′ and a second straight portion 126″. The first and second straight portions 126′, 126″ form an angle other than 0 degrees and other than 180 degrees to one another. The first straight portion 126′ contacts the inner wall 102 of the vein 100 along the line. At least a portion of the second straight portion 126″ can contact the inner wall 102 of the vein 100, or the second straight portion 126″ can be configured so that it does not contact the inner wall 102 of the vein.

The version of the energy emitting member 226 illustrated in FIG. 5B includes three straight portions, a first straight portion 226′, second straight portion 226″ and a third straight portion 226′″. The first and second straight portions 226′, 226″ form an angle other than 0 degrees and other than 180 degrees relative to one another. Similarly, the second and third straight portions 226″, 226′″ form an angle other than 0 degrees and other than 180 degrees relative to one another. The first straight portion 226′ of the energy emitting member 226 shown in FIG. 5B contacts the inner wall 102 of the vein 100 along a line.

The third version of the energy emitting member 326 illustrated in FIG. 5C also includes three straight portions 326′, 326″, 326′″. Here though, the first straight portion 326′ and the third straight portion 326′″ are not coplanar, but rather are three-dimensionally shifted. The first straight portion 326′ contacts the inside wall 102 of the vein 100 along a line. At least the distal end of the first straight portion 326′ forms an angle (other than 0 degrees and other than 180 degrees) to the longitudinal axis of the elongated member 24. The angle positions the first straight portion 326′ to be not parallel to the longitudinal axis of the emitting member 24, and the first straight portion 326′ and the longitudinal axis of the elongated member 24 do not cross three-dimensionally. When the elongated member 24 rotates, the trajectory pattern of the rotated first straight portion 326′ is one or more closed tapered tri-dimensional shapes having a spatial angle not only comprised of a round surface. The energy emitting member 26 extends continuously from the elongated member 24 in the distal direction of the extension line of the elongated member 24, and is not attached on the lateral side. This creates the rotation of the first straight portion 326′ in the predetermined space along the elongated member 24, and the rotation of the first straight portion 326′ is applied to and fits the inner wall of the vein having a decreasing diameter (an irregular diameter). The damaged inner wall 102 of the vein 100 initiates collapsing and occluding, and has somewhat different inner diameters. This shape of the energy emitting member 26 can be used to avoid curving of the first straight portion causing less contact, and it helps ensure that the first straight portion 326′ is held in contact with the inner wall 102 of the vein in the predetermined space. Thus, the rotation of the first straight portion enhances contact with the inside wall 102 of the vein along a line. The line can be straight or curved, and has a length such that contact is facilitated between the inner wall 102 of the vein 100 and the first straight portion 326′, causing the vein affected. It is easier to introduce the outer sheath because it is a line.

The first straight portion 326′ is straight in a natural state but when the force of the contact is strong enough, the first straight portion is forced to curve on the inner wall 102 of the vein 100.

When this type of energy emitting member having the first straight portion 326′ is used after mechanically or fluidically damaging the inner wall 102 (endothelium of the blood vessel), it increases the effects of occluding the vein because energy affects the damaged inner wall 102 creating more spasm.

FIG. 5D illustrates that the energy emitting member 426 can have a cylindrical shape, while FIG. 5F shows that the energy emitting member 626 can be in the shape of a cone (conical shape), with a circular base. This tapering shape shown in FIG. 5F can be beneficial in that it tends to gradually bring together the inside wall of the vein during the welding or fusing together of the inside wall of the vessel.

FIG. 5E illustrates another embodiment of the energy emitting member in which the energy emitting member 526 is generally cylindrical, but provided with a helix or helical groove as illustrated. By properly arranging the helix and rotating the energy emitting member 526, the helix or helical groove in the outer surface of the energy emitting member 526 will help automatically withdraw the energy emitting member 526 as the energy emitting member is rotated.

The central longitudinal axis of the energy emitting member 26 having a cylindrical shape or the shape of a cone (conical shape) is central to that of the elongated member 24 to fit the inner wall of the vein having a decreasing diameter (an irregular diameter). The central longitudinal axis of the energy emitting member 26 may be offset such that when the energy emitting member 26 rotates it creates a predetermined enlarged space to have more contact inside the inner wall 102 of the vein along a line. The energy emitting member 26 having a cylindrical shape or in the shape of a cone (conical shape) may be made of elastic material such that when the energy emitting member is pushed toward the inner wall 102 of the vein 100 the energy emitting member is pushed toward the inner wall 102 of the vein 100 the energy emitting member may deform to keep the energy emitting member contacting the inner wall 102 along a line.

As discussed above, the operation of the intraluminal device can involve rotating the energy emitting member, for example, by rotating the elongated member 24 to which the energy emitting member is fixed. By appropriately configuring the energy emitting member in the various ways disclosed here, the energy emitting member makes line contact with the inner wall or inner surface of the vein. That is, the energy emitting member contacts the inner wall or inner surface of the vein along a line, and then when the energy emitting member is rotated, the path of contact defined by the rotating energy emitting member is an annular band. The line contact between the energy emitting member and the inner wall surface of the vein is desirable from the standpoint of helping to avoid adherence of the energy emitting member to the inner wall surface of the vein. Examples of this line contact are illustrated in FIGS. 13-15 and 16B.

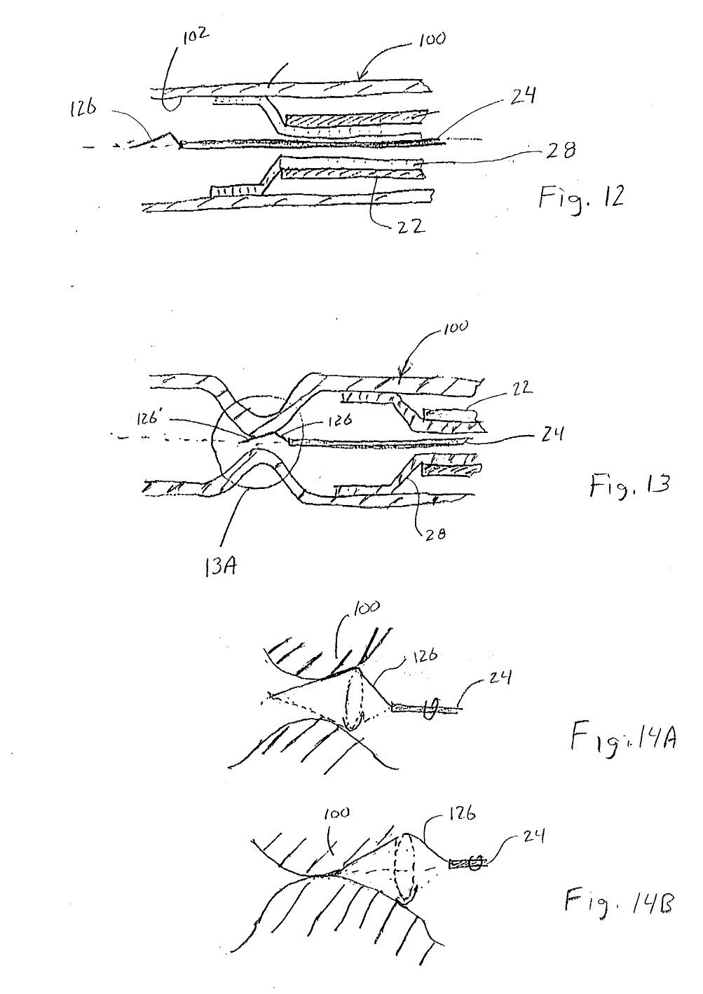

Referring initially to FIG. 12, the intraluminal device is illustrated as positioned in a vein 100. The energy emitting member 126 shown in FIG. 5A is fixed to the distal end of the elongated member 24 so that the energy emitting member 126 rotates together in unison with the elongated member 24. A portion of the vein exhibits a reduced inner diameter or inner size by virtue of the earlier contact of the self-expanding member 28 with the vein inner wall surface.

FIG. 13 illustrates that the first straight portion 126″ contacts a portion of the inner surface of the vein of reduced size. FIG. 13 illustrates that the contact between the energy emitting member 126 (the first straight portion 126″ of the energy emitting member 126) and the inner wall surface of the vein is along a line (i.e., line contact).

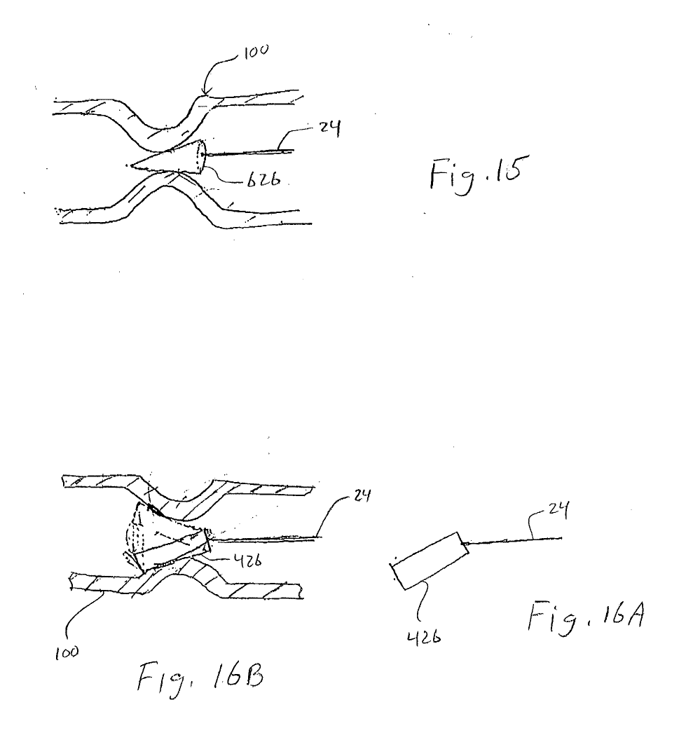

FIG. 14A illustrates the generally conical shape of revolution that occurs when the energy emitting member 126 is rotated through rotation of the elongated member 24. FIG. 14B illustrates the rotating energy emitting member 126 after the energy emitting member 126 has been moved rearwardly while applying energy, whereby protein in the vessel is heat-denatured so that the vein becomes occluded or closes as shown in FIG. 14B.

FIG. 15 illustrates the conically-shaped energy emitting member 626 shown in FIG. 5F fixed to the end of the rotatable elongated member 24 so that the energy emitting member 626 and the elongated member 24 rotate together as a unit. In this illustrated embodiment, the central axis of the elongated member 24 is coaxial with the central axis of the conically-shaped energy emitting member 626. As illustrated in FIG. 15, during rotation of the energy emitting member 626, the energy emitting member 626 contacts the inner wall surface of the vein 100 along a line of contact.

FIG. 16A illustrates the cylindrically-shaped energy emitting member 426 shown in FIG. 5D fixed to the distal end of the rotatable elongated member 24 so that the energy emitting member 426 and the elongated member 24 rotate together as a unit. In this illustrated embodiment, the central axis of the elongated member 24 and the central axis of the cylindrically-shaped energy emitting member 426 are not coaxial. That is, the central axis of the elongated member 24 and the central axis of the cylindrically-shaped energy emitting 426 intersect one another (i.e., the central axis of the elongated member 24 and the central axis of the cylindrically-shaped energy emitting member 426 intersect one another at an angle other than 0 degrees and other than 180 degrees).

FIG. 16B illustrates the energy emitting member 426 rotating as a result of rotation of the elongated member 24. As illustrated in FIG. 16B the energy emitting member 426 traces a path of movement (shape of revolution) that is generally conical. FIG. 16B also shows that the contact between the rotating energy emitting member 426 and the inner wall surface of the vein 100 is line contact.

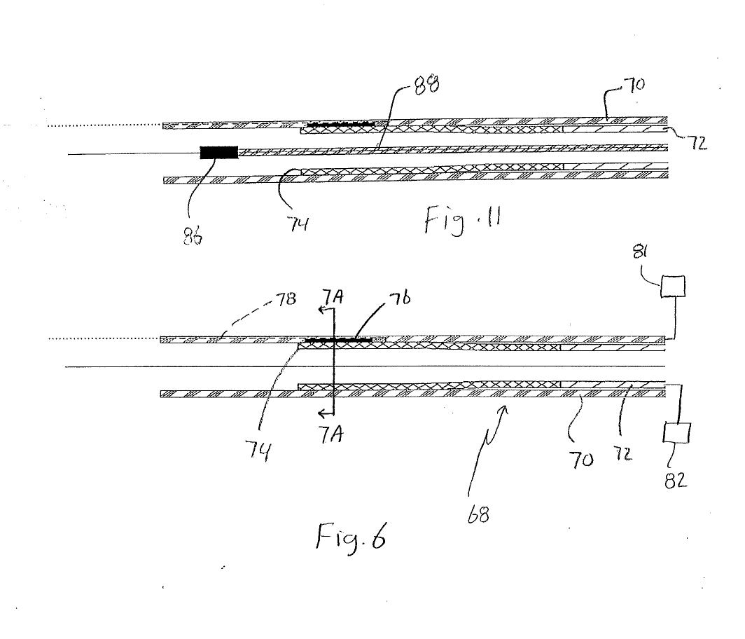

The embodiments of the intraluminal device described above utilize the self-expanding member which contacts (directly contacts) the inside wall or inside surface of the vein while being axially moved to reduce the inside diameter of the vein or collapse the vein. The embodiments also use an energy emitting member to then occlude the vein. The embodiment of the intraluminal device 68 shown in FIG. 6 may not require the energy emitting member, though as will be described below, it is possible to include the energy emitting member.

The assembly or intraluminal device 68 illustrated in FIG. 6 includes an outer sheath 70, an inner sheath 72, and a self-expanding member 74 fixed at the distal end of the inner sheath 72 so that the inner sheath 72 and the self-expanding member 74 move together as one. The outer sheath 70 is connected to a moving device 76 schematically illustrated in FIG. 6 to axially move the outer sheath 70 relative to both the inner sheath 72 and the self-expanding member 74.

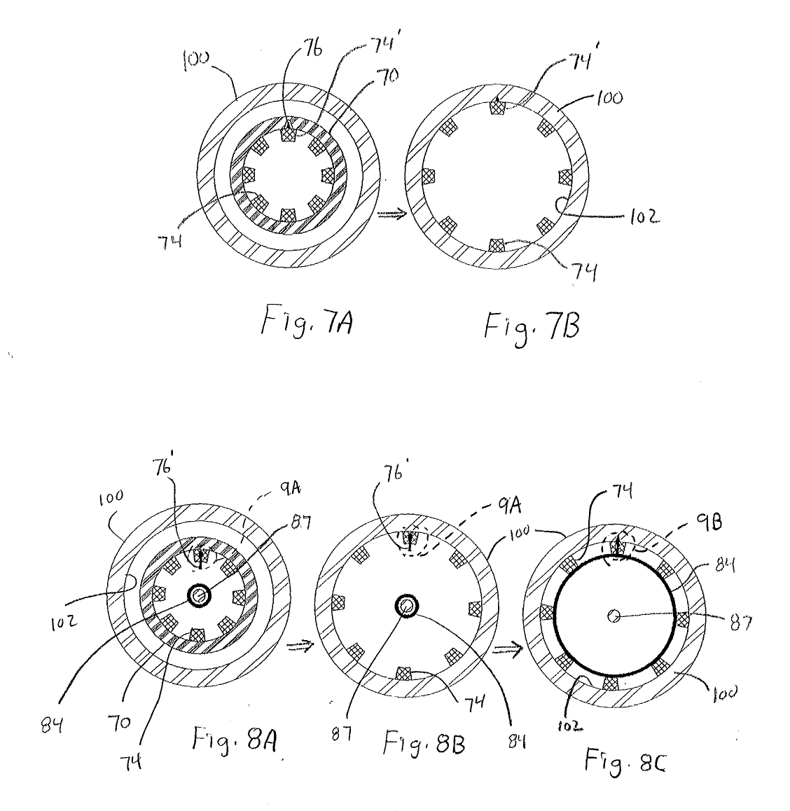

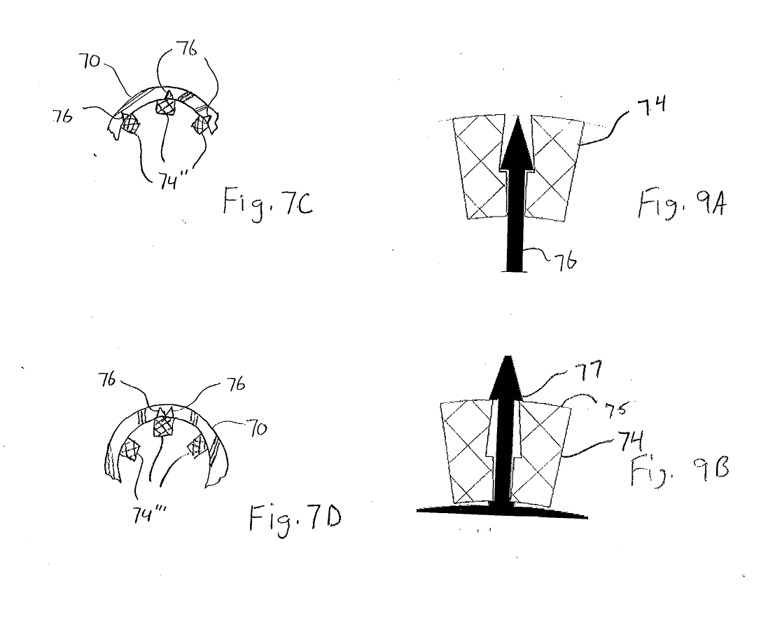

A cutting member 76 is fixed to the outside surface of the self-expanding member 74 at the distal end portion of the self-expanding member 74 as illustrated in FIG. 6 and FIG. 7A. The cutting member 76 projects outwardly away from the self-expanding member 74 and includes a pointed distal end. The tip end of the cutting member 76 extends further radially outwardly than all other portions of the self-expanding member 74. The self-expanding member may also be provided with plural cutting members 76. FIG. 7C illustrates one possible arrangement of plural cutting members 76 on the self-expanding member 74″, and FIG. 7D illustrates another possible arrangement of plural cutting members 76 on the self-expanding member 74′″. The cutting member(s) 76 is linear, extends along (parallel to) the longitudinal axis and has a triangle shape in cross-section in which one of the angles (corners) faces radially outwardly.

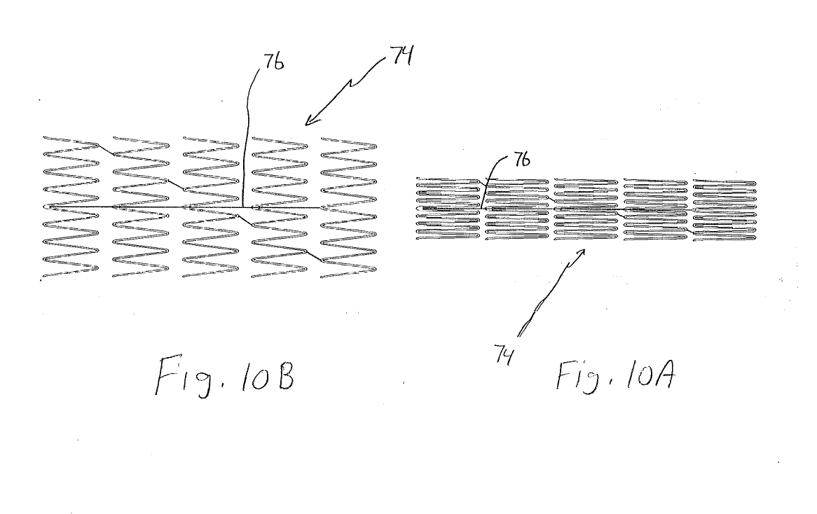

FIGS. 10A and 10B illustrate developed views of the self-expanding member used in the assembly or intraluminal device 68 illustrated in FIG. 6. That is, FIGS. 10A and 10B depict the self-expanding member 74 as though it was cut along its axial extent and then laid flat. FIG. 10A illustrates the self-expanding member 74 before expansion, while FIG. 10B illustrates the self-expanding member 74 after outward expansion. The self-expandable member 74 is comprised of a plurality of axially arranged annular (ring-shaped) wavy-shaped members. The annular wavy-shaped members are disposed so that the axis of each annular wavy-shaped member is arranged along a common line. That is, all of the annular wavy-shaped members are coaxial. Each annular wavy-shaped member has a plurality of alternating peaks and valleys one after the other in the circumferential direction of the ring. The self-expandable member 74 is configured to connect or contact the peaks of the annular wavy-shaped members longitudinally. When the annular wavy-shaped members are expanded radially outwardly, the peaks of the self-expandable member 74 are positioned to contact the inner wall of the vein. The cutting member(s) 76 is fixed to the peaks that extend along the longitudinal axis of the outside surface of the self-expanding member 74 at the distal end portion of the self-expanding member 74 as illustrated in FIG. 10A, 10B. That is, the cutting member(s) 76 extend along the axial extent of the axially arranged annular wavy-shaped members forming the self-expanding member 74 and span the axially aligned peaks in axially adjacent annular wavy-shaped members.

As illustrated, the cutter member 76 extends along the axial extent of the self-expanding member 74. In the unexpanded state and the expanded state, the length and the angle or orientation of the cutting member 76 does not change. In both the unexpanded state shown in FIG. 10A and in the expanded state shown in FIG. 10B, the cutting member is parallel to the central axis of the self-expanding member 74. It is thus relatively easy for the cutter member to follow the self-expandable member without the change of the cutter member shape.

As shown in FIG. 6, the inner surface of the outer sheath 70 is provided with a groove 78 represented by the dotted line. This groove 78 receives the cutting member 76 which projects away from (i.e., is upstanding with respect to) the outer surface of the self-expanding member 74 so that relative movement between the outer sheath 70 and the inner sheath 72/self-expanding member 74 is not hindered by the presence of the cutting member 76.

In use, the assembly or intraluminal device 68 is inserted into the vein of interest (e.g., the varicose vein to be treated). The intraluminal device is moved within the vein 100 to position the distal end of the intraluminal device at the target site or treatment site. In this condition, the cross-section of the assembly or intraluminal device 68 in the region of the cutting member 76 is as shown in FIG. 7A which is a cross-section at the section line 7A-7A in FIG. 6.

Next, the moving device 81 is operated to axially move or withdraw the outer sheath 70 relative to the inner sheath 72/self-expanding member 74. The outer sheath 70 is proximally moved to expose the distal end of the self-expanding member 74 as discussed above.

FIG. 7A illustrates the outer sheath 70 in covering relation to the self-expanding member 74. FIG. 7B illustrates the distal end portion of the device when the outer sheath 70 is withdrawn or moved proximally relative to the inner sheath 72/self-expanding member 74. The relative movement between the outer sheath 70 and the inner sheath 72/self-expanding member 74 causes the self-expanding member to extend or be positioned distally beyond the distal-most end of the outer sheath 70 so that the distal end portion of the self-expanding member moves into contact with the inner wall or inner surface 102 of the vein 100.

The rotating and moving device 82 is operatively connected to the inner sheath 72 to rotate the inner sheath 72 as well as the self-expanding member 74. When the self-expanding member 74 is in contact with the inner wall 102 of the vein 100 as shown in FIG. 7B the rotating and moving device 82 is operated to rotate the self-expanding member 74. As a result, the cutting member 76 damages the inner surface or inner wall 102 of the vein 100, thus forming blood clots that occlude the vein 100 (the lumen in the vein).

The outer sheath 70 continues to be axially moved in the proximal direction through operation of the moving device 81 while the inner sheath 72 is also axially moved in the proximal direction and while the inner sheath 72 is being rotated through operation of the rotating and moving device 82. The cutting member 76 thus damages the inner surface or inner wall 102 of the vein 100 along the axial extent of the vein, thus forming blood clots along the axial extent of the vein. After the outer sheath 70 and inner sheath 72 have been moved in the rearward proximal direction to the extent necessary to cause the cutting member 76 to act on the desired extent of the vein, operation of the moving device 81, and operation of the rotating and moving device 82, are stopped, and the assembly or device 68 is withdrawn from the vein.

FIGS. 8A-8C illustrate a variation on the embodiment shown in FIG. 6 and FIGS. 7A-7B. The variation shown in FIG. 8A includes the outer sheath 70 and the self-expanding member 74 fixed to the inner sheath 72. The device is also provided with a cutting member 76′. This cutting member 76′ is illustrated in more detail in FIG. 9A. The cutting member 76′ is positioned in a slot in the self-expanding member 74 and is movable from the position shown in FIG. 9A to the position shown in FIG. 9B. When the self-expanding member 74 is covered by the outer sheath 70 as illustrated in FIG. 8A, the cutting number 76′ is positioned in a manner illustrated in FIG. 9A in which the tip end of the cutting member 76′ is radially inwardly of the outer surface of the self-expanding member 74. The cutting member 76 has a tapered or sharpened distal end, an elongated shaft, and a flattened proximal end having a larger diameter than the elongated shaft and the tapered distal end. The distal end and the proximal end of the cutting member 76 are connected by the shaft having a decreased diameter compared to the distal end and the proximal end. The distal end of the cutting member 76 is accommodated in a recess or hole in the surface of the self-expanded member 74 as shown in FIG. 9A. In the state shown in FIG. 9A, the most distal end of the cutting member 76 does not protrude from (i.e., is recessed relative to or at the same level as) the outer sheath or outer surface of the self-expanded member 74. When the proximal end of the cutting member 76 is pushed radially outwardly, the flattened portion having a larger diameter is pushed outwardly and the cutting member 76 is forced to move radially outwardly. When the flattened portion of the cutting member 76 contacts the inner surface (opposite surface) of the self-expanded member 74, the cutting member 76 stops moving radially outwardly, and the most distal end of the cutting member 76 protrudes from (i.e., radially outwardly beyond) the outer surface of the self-expanded member 74.

As illustrated in FIG. 8A, a centrally located expandable member 84 is positioned inside the outer sheath 70 and the self-expanding member 74. In the illustrated embodiment, the expandable member 84 is coaxial with the outer sheath 70 and the inner sheath 72. In the position illustrated in FIG. 8A in which the self-expanding member 74 is housed and covered by the outer sheath 70, the expandable member 84 is in the non-expanded position shown in FIG. 8A. An inner tube 87 is also centrally positioned in the expandable member 84.

When the outer sheath 70 is moved proximally relative to the inner sheath 72/self-expanding member 74, the self-expanding member 74 is positioned distally beyond the distal end of the outer sheath 70 so that the self-expanding member 74 is exposed. The self-expanding member 74 thus automatically self-expands outwardly into contact with the inner wall or inner surface 102 of the vein as illustrated in FIG. 8B. In this retracted position, the cutting member 76′ remains in the position illustrated in FIG. 9A. FIG. 9A shows that the end (proximal end) of the cutting member 76 opposite the tip end extends further radially inwardly than the inner surface of the self-expanding member 74 when the cutting member 76′ is in the retracted position.

Next, as illustrated in FIG. 8C, the expandable member 84 is radially outwardly expanded to contact the proximal end of the cutting member 76′, thus radially outwardly pushing the cutting member 76′ into contact with the inner surface or inner wall 102 of the vein 100 as shown in FIG. 8C. At this time, the cutting member 76′ takes the position shown in FIG. 9B. As an example, the expandable member 84 may be a conventional balloon catheter (either over the wire type or rapid exchange type) having a balloon (expandable member 84). A balloon catheter has an outer tube and an inner tube, and a balloon is placed between the distal end of the inner tube and the distal end of the outer tube, between which creates a lumen for delivering expanding fluid. When a fluid is delivered to the balloon through the lumen, the balloon (expandable member 84) is expanded to thus radially outwardly push the cutting member 76′ into contact with the inner surface or inner wall 102 of the vein 100.

FIG. 9B shows that the cutting member 76′ can be provided with an enlarged head portion 77 that engages (contacts) the outer surface 75 of the self-expanding member 74. The cutting member 76′ is thus retained in the extended position shown in FIG. 9B. During subsequent rotation and withdrawal of the inner sheath 72 and the self-expanding member 74 (and possibly the expandable member 84), the cutting member 76′ damages the inner surface or inner wall 102 of the vein 100, thus forming blood clots as discussed above.

As explained above, the embodiment of the device or assembly 68 illustrated in FIG. 6 may not require an energy-emitting member similar to the energy-emitting member used in the earlier embodiments of the device. Nevertheless, as illustrated in FIG. 11, it is possible, if desired, to include an energy emitting member 86 fixed to the distal end of an elongated member 88. The energy emitting member 86 would be used in a manner similar to that discussed above. That is, during rotation and withdrawal of the inner sheath 72 and the self-expanding member 74, the elongated member 88 and the energy emitting member 86 would also be withdrawn. Energy supplied to the energy emitting member 86 would be applied to the inner surface of the vein to help occlude the vein by, for example, ablating or cauterizing the inner wall of the vein.

The detailed description above describes features and aspects of examples of embodiments of a vein treatment method and assembly/intraluminal device. The present invention is not limited, however, to the precise embodiments and variations described. Various changes, modifications and equivalents could be effected by one skilled in the art without departing from the spirit and scope of the invention as defined in the appended claims. It is expressly intended that all such changes, modifications and equivalents which fall within the scope of the claims are embraced by the claims.

What is claimed is:

1. A method of treating a varicose vein comprising:
inserting an assembly into the vein, the assembly being comprised of: an outer sheath that includes a contact member and possesses a distal end; a catheter movably positioned inside the outer sheath and possessing a distal end at which is located a self-expanding member, the self-expanding member possessing a distal end; and an elongated body positioned inside the catheter and possessing a distal end at which is located an energy emitting member, the energy emitting member possessing a distal end;
moving the assembly to a treatment site in the vein;
moving the contact member into contact with an inner wall of the vein;
withdrawing the outer sheath relative to the catheter and the self-expanding member while the contact member remains in contact with the inner wall of the vein to cause a first spasm in the vein that decreases an inner diameter of the vein;
the withdrawing of the outer sheath relative to the catheter and the self-expanding member causing the distal end of the self-expanding member to be located distally beyond the distal end of the outer sheath so that the self-expanding member self-expands outwardly into contact with the inner wall of the vein;
moving the catheter and the self-expanding member in a proximal direction relative to the vein while the self-expanding member is in contact with the inner wall of the vein to cause a second spasm in the vein that further decreases the inner diameter of the vein so that the vein collapses;
applying energy from the energy emitting member to the inner wall of the collapsed vein to cause the vein to uniformly and immediately occlude; and
withdrawing the assembly from the vein.

2. The method according to claim 1, wherein the self-expanding member is movable relative to the energy emitting member.

3. The method according to claim 1, wherein the self-expanding member is fixed relative to the energy emitting member.

4. The method according to claim 1, wherein the energy emitting member is positioned distally beyond the distal end of the self-expanding member throughout the insertion of the assembly into the vein and the movement to the treatment site.

5. The method according to claim 1, wherein the energy emitting member is positioned distally beyond the distal end of the self-expanding member throughout an entirety of the withdrawal of the outer sheath relative to the catheter and the self-expanding member.

6. The method according to claim 1, wherein the self-expanding member possesses a distal-most end and the energy emitting member possesses a distal-most end, a distance from the distal-most end of the self-expanding member to the distal-most end of the energy emitting member being 1 mm-50 mm.

7. The method according to claim 1, wherein the contacting member is an expandable member on an outer surface of the outer sheath, the contact member being moved into contact with the inner wall of the vein by inflating the expandable member.

8. The method according to claim 1, wherein the contacting member and the self-expanding member are made of different materials.

9. The method according to claim 1, wherein the elongated body includes an aspiration port positioned proximally of the energy emitting member.

10. The method according to claim 1, wherein the self-expanding member possesses a length of 10 mm-200 mm.

11. A method of treating a varicose vein comprising:
inserting an assembly into the vein, the assembly being comprised of: an outer sheath that includes a contact member and possesses a distal end; a self-expanding member positioned inside the outer sheath, the self-expanding member possessing a distal end and being outwardly expandable when the distal end of the self-expanding member is exposed distally beyond the distal end of the outer sheath; and an energy emitting member;
moving the assembly in the vein to position the assembly at a desired place in the vein;
contacting an inner wall of the vein with the contact member;
moving the outer sheath relative to the inner wall of the vein while the contact member is in contact with the inner wall of the vein to produce a first spasm in the vein that decreases an inner diameter of the vein;
relatively moving the outer sheath and the self-expanding member while the contact member is in contact with the inner wall of the vein to expose the distal end of the self-expanding member distally beyond the distal end of the outer sheath such that the self-expanding member self-expands outwardly into contact with an inner wall of the vein at a position distal of a position at which the contact member is in contact with the inner wall of the vein;
decreasing an inner diameter of the vein and causing the vein to collapse by moving the expanded self-expanding member relative to the vein while the expanded self-expanding member is in contact with the inner wall of the vein; and
occluding the vein by applying energy from the energy emitting member to the inner wall of the vein which has collapsed.

12. The method according to claim 11, wherein the self-expanding member and the energy emitting member are movable relative to one another.

13. The method according to claim 11, wherein the self-expanding member and the energy emitting member are fixed in position relative to one another.

14. The method according to claim 11, wherein the self-expanding member possesses a distal-most end and the energy emitting member possesses a distal-most end, a distance from the distal-most end of the self-expanding member to the distal-most end of the energy emitting member being 1 mm-50 mm.

15. The method according to claim 11, wherein the contacting member is an expandable member on an outer surface of the outer sheath, the contact member being moved into contact with the inner wall of the vein by inflating the expandable member.

16. The method according to claim 11, wherein the contacting member and the self-expanding member are made of different materials.

17. The method according to claim 11, wherein the elongated body includes an aspiration port positioned proximally of the energy emitting member.

18. The method according to claim 11, wherein the contacting member is moved into contact with the inner wall of the vein by outwardly expanding the contact member.

19. The method according to claim 11, wherein the self-expanding member possesses a length of 10 mm-200 mm.

20. The method according to claim 11, wherein the energy emitting member is positioned distally beyond the distal end of the self-expanding member throughout the insertion of the assembly into the vein and the movement to the treatment site.
